(19) United States
(12) Patent Application Publication
(10) Pub. No.: US 2023/0309901 A1
(43) Pub. Date: Oct. 5, 2023

(54) IN-EAR BALANCE DETECTION SYSTEM

(71) Applicant: Change Healthcare Holdings, LLC, Nashville, TN (US)

(72) Inventor: David Alan Wright, Nashville, TN (US)

(21) Appl. No.: 17/704,366

(22) Filed: Mar. 25, 2022

(51) Int. Cl. G06F 3/16; A61B 5/00; A61B 17/22

(52) U.S. Cl. CPC A61B 5/4023; A61B 5/0002; A61B 5/6817; A61B 5/6847; A61B 5/741; A61B 5/742; A61B 17/22004; G06F 3/16; A61B 2562/0219

(57) ABSTRACT

A balance detection and correction system includes at least one sensor positioned in an ear of a user, a processor, and a memory having instructions stored thereon that, when executed by the processor, causes the processor to perform operations including continuously tracking a position of the user’s head based on first data obtained from the first sensor, comparing the position of the user’s head to second data obtained from a second sensor positioned on the user’s body to detect an imbalance in the user’s posture or movements, and initiating a response action responsive to the detection of an imbalance.

### BACKGROUND

The present disclosure relates generally to a wearable or implantable device for detecting imbalances in human movement.

Balance disorders, such as vertigo, affect thousands of people around the world and can have a detrimental impact on one’s quality of life. One of the more common causes of vertigo, benign paroxysmal positional vertigo (BPPV), affects more than 200,000 people in the United States alone each year, for example. BPPV occurs when tiny calcium crystals called otoconia come loose from their normal location on the utricle, a sensory organ in the inner ear. Detached otoconia can flow freely in the fluid-filled spaces of the inner ear, including the semicircular canals (SCC) that sense the rotation of the head, causing balance issues and dizziness. While there is often no singular or specific cause for BPPV, events such as head trauma, suddenly and rough movements, high intensity exercise, and inner-ear infections, among others, may initiate a sudden onset of symptoms.

In some cases, particularly with mild BPPV or other balance-affecting conditions, symptoms are not always immediately obvious or may be mild enough that an affected person does not notice. For example, BPPV may result in slight imbalances that can cause the affected person to unknowingly deviate from their path when walking, which can then cause the person to run into objects, stumble, etc. As another example, BPPV may cause balance issues that, in turn, cause the affect person to fall or feel unsteady. While there exists a number of well-known exercises for treating the symptoms of vertigo (e.g., BPPV), these exercises are generally only performed after the onset of obvious symptoms. However, there is a lack of technology that can readily identify imbalances due to BPPV and other balance-related conditions, and that can provide treatment options for a user.

### SUMMARY

One implementation of the present disclosure is a balance detection and correction system that includes at least one sensor positioned in an ear of a user, a processor, and a memory having instructions stored thereon that, when executed by the processor, causes the processor to perform operations including continuously tracking a position of the user’s head based on first data obtained from the first sensor, comparing the position of the user’s head to second data obtained from a second sensor positioned on the user’s body to detect an imbalance in the user’s posture or movements, and initiating a response action responsive to the detection of an imbalance.

In some embodiments, the at least one sensor is worn in the ear of the user or implanted in the ear of the user.

In some embodiments, the at least one sensor includes at least one of a gyroscope, an accelerometer, or an inertial measurement unit (IMU).

In some embodiments, comparing the position of the user’s head to the second data includes calculating a center position and a trajectory of the user based on the second data, and comparing the first data to the center position and the trajectory to detect the imbalance.

In some embodiments, the system further includes a speaker positioned in the ear of the user, and the speaker and the at least one sensor are contained within a single housing.

In some embodiments, the response action includes emitting, by the speaker, at least one of a tone or a voice prompt that indicates to the user that the imbalance is detected.

In some embodiments, the response action includes emitting, by the speaker, ultrasonic sounds waves configured to break apart calcium crystals in the user’s ear.

In some embodiments, the response action includes causing a user device associated with the user to display a notification.

In some embodiments, the response action includes causing a user device associated with the user to display a prompt to perform one or more exercises for correcting the imbalance.

Another implementation of the present disclosure is a method of detecting imbalances in human motion which includes continuously receiving first data from a first sensor worn or implanted in an ear of a user, the first data indicating a position and motion of the user’s head, receiving second data from a second sensor positioned on the body of the user, the second data indicating a position and motion of the user’s body, comparing the first data and the second data to detect an imbalance in the user’s movements, and initiating a response action responsive to the detection of an imbalance.

In some embodiments, comparing the first and the second data further includes calculating a center position and a trajectory of the user based on the second data and comparing the first data to the center position and the trajectory to detect an imbalance.

In some embodiments, each of the first sensor and the second sensor include at least one of a gyroscope, an accelerometer, or an inertial measurement unit (IMU).

In some embodiments, the response action includes emitting, by a speaker positioned in the user’s ear, at least one of a tone or a voice prompt that indicates to the user that the imbalance is detected.

In some embodiments, the response action includes emitting, by a speaker positioned in the user’s ear, ultrasonic sounds waves configured to break apart calcium crystals in the user’s ear.

In some embodiments, the response action includes causing a user device associated with the user to display a notification.

In some embodiments, the response action includes causing a user device associated with the user to display a prompt to perform one or more exercises for correcting the imbalance.

Yet another implementation of the present disclosure is an in-ear balance detection device including a gyroscope for detecting an orientation of a user’s head, an accelerometer for detecting movement of the user’s head, a wireless transceiver configured to wirelessly communicate data with a remote processing device, wherein the wireless transceiver transmits data from the gyroscope and the accelerometer, and wherein the wireless transceiver receives control signals from the remote processing device responsive to detection of an imbalance in the user’s movements based on the data from the gyroscope and the accelerometer, and a speaker configured to emit auditory alerts responsive to a received control signal, wherein the auditory alerts indicate to the user that an imbalance is detected.

In some embodiments, the device is worn in an ear of the user.

In some embodiments, the device implanted in the ear of the user.

In some embodiments, the device further includes an inertial measurement unit (IMU), wherein the gyroscope and the accelerometer are part of the IMU.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a block diagram of an in-ear balance detection device, according to some embodiments.

FIGS. 2A-2C are diagrams illustrating various configurations of the in-ear balance detection device of FIG. 1, according to some embodiments.

FIG. 3 is a block diagram of a balance detection system that includes the in-ear balance detection device of FIG. 1, according to some embodiments.

FIG. 4 is a flow diagram of a process for detecting imbalances in a user’s movements, according to some embodiments.

FIGS. 5A and 5B are example user interfaces that allow a user to select and perform various exercise for correcting vertigo, according to some embodiments.

### DETAILED DESCRIPTION

Referring generally to the figures, a balance detection system is shown which includes an in-ear device for detecting imbalances in a user’s posture and/or movements. In particular, the in-ear device may be wearable by a user and/or implantable in at least one of the user’s ears, and may include various sensors (e.g., a gyroscope, an accelerometer, etc.) that detect the position and movement of the user’s head. The in-ear device may communicate wirelessly with a remote computing device, such as the user’s smartphone, which processes data from the in-ear device to detect imbalances. When an imbalance is detected, a number of different response actions may be initiated. In some cases, the in-ear device may emit an audible tone or voice prompt that alerts the user to the imbalance. For example, if it is detected that the user is drifting in one direction when walking, a voice prompt may be emitted that alerts the user to prevent the user from running into objects, etc. In some embodiments, the in-ear device is configured to emit high-frequency (e.g., ultrasonic) soundwaves, or pulses, that can break up the otoconia that may cause certain symptoms of, for example, BPPV. Additional features and advantages are described in greater detail below.

### In-Ear Balance Detection Device

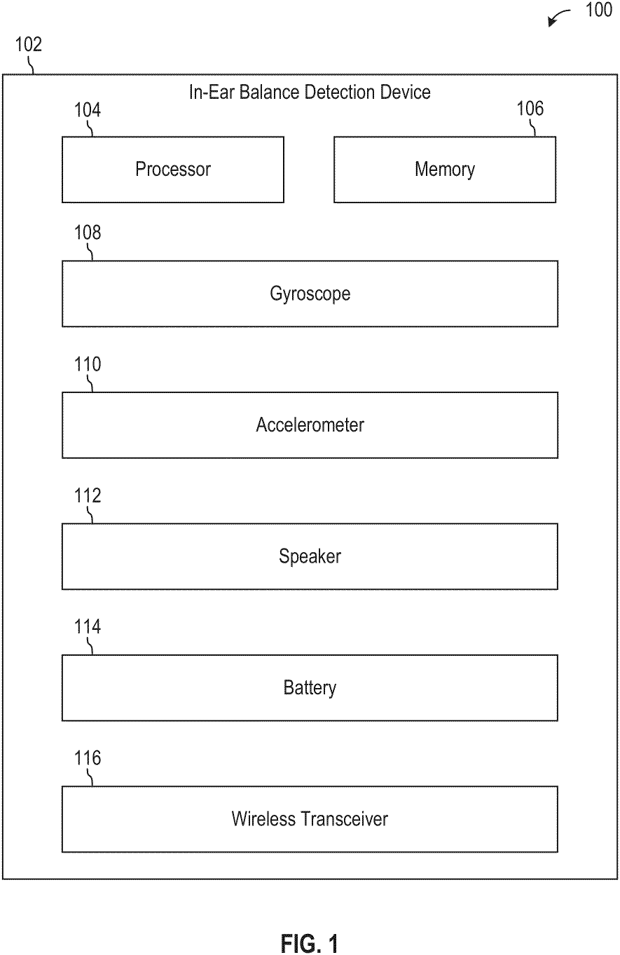

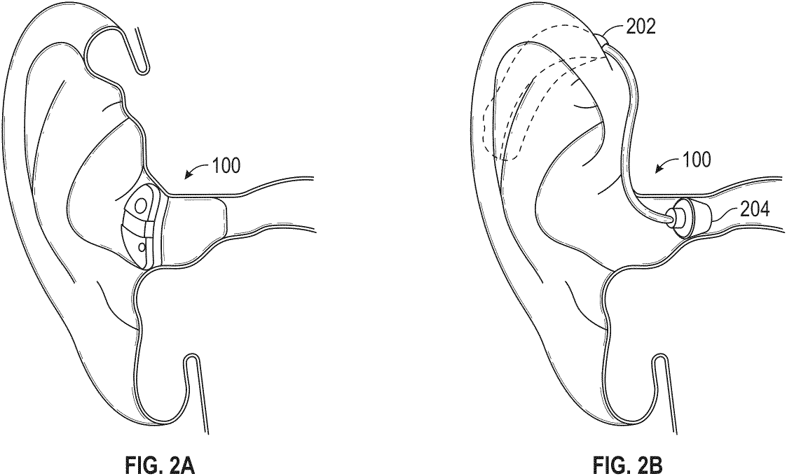

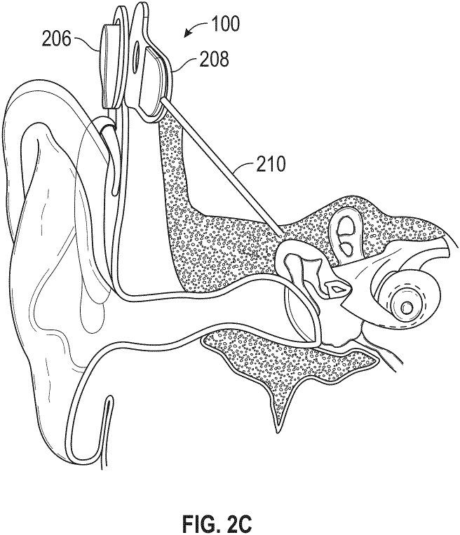

Turning first to FIG. 1, a block diagram of an in-ear balance detection device 100 is shown, according to some embodiments. As will be described in greater detail below, device 100 is generally configured to be worn in the ear of a user or, in some cases, may be implanted in the user’s ear. In some embodiments, a user may use (e.g., wear or have implanted) one of device 100 in each ear. As shown, device 100 includes a housing 102 which encloses a number of internal components. Housing 102 may be formed of any suitable material depending on whether device 100 is implanted or worn. For example, in a wearable configuration (e.g., as shown in FIGS. 2A and 2B), housing 102 may be formed of plastic, acrylic, silicone, or the like. In an implantable configuration, housing 102 may be formed of any biomaterial that is safe for implantation, such as certain plastics, metal (e.g., titanium), etc.

Enclosed within housing 102 is a processor 104 and a memory 106. Processor 104 can be a general-purpose processor, an application specific integrated circuit (ASIC), one or more field programmable gate arrays (FPGAs), a group of processing components, or other suitable electronic processing components. In some embodiments, processor 104 is configured to execute program code stored on memory 106 to cause device 100 to perform one or more operations. Memory 106 can include one or more devices (e.g., memory units, memory devices, storage devices, etc.) for storing data and/or computer code for completing and/or facilitating the various processes described in the present disclosure. In some embodiments, memory 106 includes tangible, computer-readable media that stores code or instructions executable by processor 306. Tangible, computer-readable media refers to any media that is capable of providing data that causes device 100 to operate in a particular fashion. Example tangible, computer-readable media may include, but is not limited to, volatile media, non-volatile media, removable media and non-removable media implemented in any method or technology for storage of information such as computer readable instructions, data structures, program modules or other data.

Accordingly, memory 106 can include random access memory (RAM), read-only memory (ROM), hard drive storage, temporary storage, non-volatile memory, flash memory, optical memory, or any other suitable memory for storing software objects and/or computer instructions. Memory 106 can include database components, object code components, script components, or any other type of information structure for supporting the various activities and information structures described in the present disclosure. Memory 106 can be communicably connected to processor 104, such as via processing circuit (not shown), and can include computer code for executing (e.g., by processor 104) one or more processes described herein. While shown as individual components, it will be appreciated that processor 104 and/or memory 106 can be implemented using a variety of different types and quantities of processors and memory. For example, processor 104 may represent a single processing device or multiple processing devices. Similarly, memory 106 may represent a single memory device or multiple memory devices. It should also be appreciated that, in some embodiments, processor 104 and/or memory 106 are optional for device 100. In some such embodiments, device 100 may not include any processing or memory devices and may instead transmit data to remote devices for processing, which can lower power consumption.

Device 100 is further shown to include a gyroscope 108 configured to detect or measure an orientation and/or angular velocity of device 100, which in turn can indicate an orientation and/or angular velocity of the user’s head (e.g., when device 100 is worn or implanted). In some embodiments, gyroscope 108 is a microelectromechanical systems (MEMS) gyroscope or gyrometer. In some embodiments, gyroscope 108 is a 3-axis gyroscope. In conjunction, an accelerometer 110 may measure linear acceleration and/or movement of device 100, which in turn can indicate an acceleration and/or movement of the user’s head. In some embodiments, accelerometer 110 is a 3-axis accelerometer. In some embodiments, rather than individual components as shown in FIG. 1, device 100 may include an inertial measurement unit (IMU) which functions as, and/or includes both of, gyroscope 108 and accelerometer 110. In such embodiments, the IMU may measure each of the specific force, angular rate, and orientation of device 100.

As shown, device 100 can also include a speaker 112 configured to emit sound (e.g., as soundwaves). Speaker 112 may be configured, in particular, to emit sound into a user’s ear (e.g., into the ear canal). In some embodiments, speaker 112 is configured to emit one or both of ultrasonic frequencies (e.g., above 20 kHz) and frequencies in the normal hearing range of humans (e.g., 20 Hz to 20 kHz). In some embodiments, as described in detail below, speaker 112 is configured to emit ultrasonic pulses that can break apart calcium crystals (e.g., otoconia) in the user’s ear, which are known to cause certain symptoms of vertigo.

In some embodiments, device 100 includes a battery 114 configured to provide electrical energy to any of the internal components described herein. Battery 114 may be any suitable type of rechargeable or replaceable battery, such as a lithium-ion battery or even a zinc-air battery (e.g., a disposable hearing aid battery). Device 100 is further shown to include a wireless transceiver 116 configured to wirelessly communicate (e.g., transmit and receive) data with one or more remote devices, such as a personal computing device associated with the user (e.g., a smartphone). In general, wireless transceiver is a short-range wireless transceiver. For example, wireless transceiver 116 may be or include one or more of a Bluetooth^{®}, ultrawide band (UWB), ZigBee, or Wi-Fi transceiver.

Referring now to FIGS. 2A-2C, diagrams illustrating various configurations of device 100 are shown, according to some embodiments. Turning first to FIG. 2A, a first wearable configuration of device 100 is shown. Specifically, in this configuration, device 100 is shown to be removably inserted into the canal of a user’s ear, similar to a traditional hearing aid. In such embodiments, housing 102 may be formed into an ergonomic shape that coincides with the user’s ear canal and all of the internal components of device 100 may be included in housing 102 (e.g., positioned in the user’s ear canal). In another wearable configuration, shown in FIG. 2B, a body 202 of device 100 is positioned behind the user’s ear. In some such embodiments, body 202 is comprised of housing 102 and the various internal components of device 100, as described above. Further, an earpiece 204 may be inserted into the user’s ear canal. In this manner, device 100 may still determine the position and/or movement of the user’s head (e.g., due to the placement of first portion 202) while sounds from speaker 112 may be routed into the user’s ear canal by earpiece 204.

In yet another configuration, shown in FIG. 2C, at least a portion of device 100 is implantable into the ear and/or under the skin of the user. In some such embodiments, a body of device 100 is positioned behind the user’s ear (e.g., similar to FIG. 2B) and is electrically coupled to a transmitter 206, which wirelessly communicates with a receiver 208. In some embodiments, the body of device 100 includes housing 102 and the various internal components of device 100, as described above. In some embodiments, receiver 208 is implanted under the skin of the user, similar to a traditional cochlear implant. Rather than emitting sounds via a speaker (e.g., speaker 112), receiver 208 may stimulate the user’s cochlear nerve or another part of the user’s inner-ear via an electrode 210. While various different configurations of device 100 are shown for reference, it should also be appreciated that device 100 may be worn on or over the user’s ears (e.g., similar to headphones).

### Balance Detection System

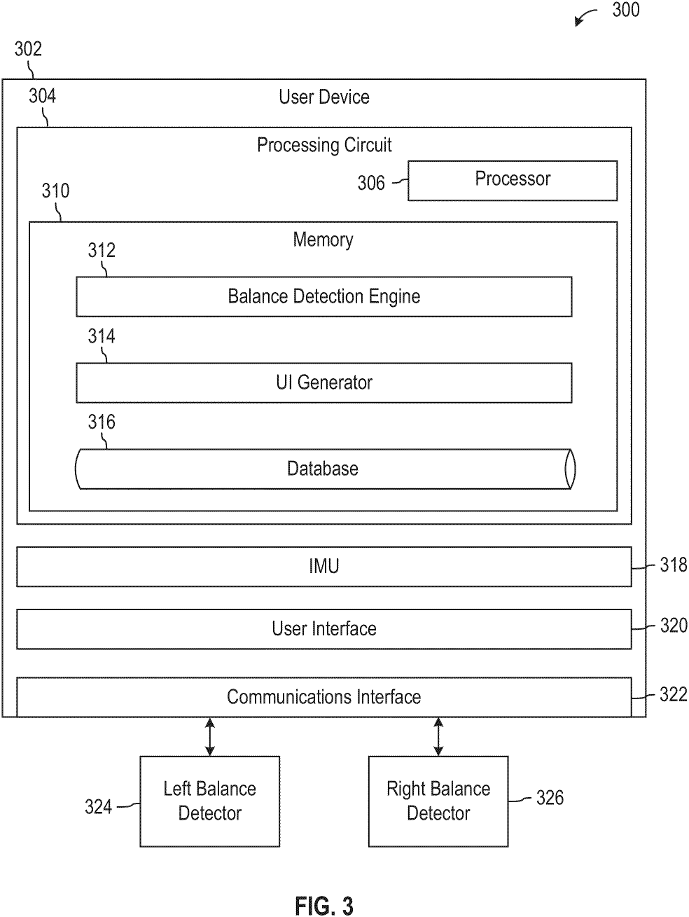

Referring now to FIG. 3, a block diagram of a balance detection system 300 is shown, according to some embodiments. As described above, system 300 generally functions to detect imbalances in a user’s movements (e.g., while walking or standing) and can automatically provide or suggest corrective actions. As described herein, an imbalance can refer to any type of balance issue experienced by a human when sitting, standing, moving (e.g., walking), lying down, etc. For example, system 300 may detect that a user is drifting in one direction when walking, which may indicate an imbalance. In another example, system 300 may detect that the user has maintained a constant or nearly-constant position (e.g., sitting) for an extended period of time, which may trigger vertigo symptoms; thus, system 300 may provide the user with an alert and/or may suggest exercises to prevent the onset of symptoms. As yet another example, system 300 may detect that the user is experience dizziness or an imbalance when standing, which can be detected from the user’s head movements (e.g., the user is leaning to one side or is moving their head in an unusual manner).

As shown, system 300 includes a user device 302 which may represent any computing device that is operable by a user. For example, user device 302 may be a personal computer, a laptop, a tablet, a smartphone, a smart watch, or the like. In some embodiments, user device 302 is a dedicated controller or processing device. However, in general, user device 302 is portable and/or is configured to be carried the user. For example, user device 302 may be a smartphone that is carried in the user’s pocket or may be a smart watch carried on the user’s wrist. User device 302 may receive data from one or both of a left balance detector 324 and a right balance detector 326. As described herein, left balance detector 324 and right balance detector 326 may each be device 100. In other words, left balance detector 324 and right balance detector 326 may be separate devices that are the same as, or functionally equivalent to, device 100. In general, left balance detector 324 represents a first one of device 100 worn or implanted in the user’s left ear, and right balance detector 326 represents a second one of device 100 worn or implanted in the user’s right ear.

In some embodiments, user device 302 includes a communications interface 322 for transmitting data to, and/or receiving data from, left balance detector 324 and right balance detector 326. While not shown, communications interface 322 can also provide means for user device 302 to communicate with any other remote devices. In general, communications interface 322 can be or can include a wireless communications interface (e.g., antenna(s), transmitter(s), receiver(s), transceiver(s), etc.) for conducting data communications. It should be appreciated that communications interface 322 may include multiple different interfaces (e.g., multiple different transceivers). In some embodiments, communications interface 322 facilitates a direct wireless connection (e.g., via a short-range wireless connection). In some embodiments, communications interface 322 facilitates wireless communications via a network (e.g., a WAN, the Internet, a cellular network, etc.). For example, communications interface 322 can include a WiFi transceiver for communicating via a wireless communications network. In another example, communications interface 322 may include cellular or mobile phone communications transceivers. However, in general, communications interface 322 includes at least a short-range wireless transceiver, such as one or more of a Bluetooth°, ultrawide band (UWB), ZigBee, or Wi-Fi transceiver.

User device 302 is shown to include a processing circuit 304 that includes a processor 306 and a memory 310. Processor 306 can be a general-purpose processor, an application specific integrated circuit (ASIC), one or more field programmable gate arrays (FPGAs), a group of processing components, or other suitable electronic processing components. In some embodiments, processor 306 is configured to execute program code stored on memory 310 to cause user device 302 to perform one or more operations. Memory 310 can include one or more devices (e.g., memory units, memory devices, storage devices, etc.) for storing data and/or computer code for completing and/or facilitating the various processes described in the present disclosure. In some embodiments, memory 310 includes tangible, computer-readable media that stores code or instructions executable by processor 306. Tangible, computer-readable media refers to any media that is capable of providing data that causes the controller 110 (i.e., a machine) to operate in a particular fashion. Example tangible, computer-readable media may include, but is not limited to, volatile media, non-volatile media, removable media and non-removable media implemented in any method or technology for storage of information such as computer readable instructions, data structures, program modules or other data.

Accordingly, memory 310 can include random access memory (RAM), read-only memory (ROM), hard drive storage, temporary storage, non-volatile memory, flash memory, optical memory, or any other suitable memory for storing software objects and/or computer instructions. Memory 310 can include database components, object code components, script components, or any other type of information structure for supporting the various activities and information structures described in the present disclosure. Memory 310 can be communicably connected to processor 306, such as via processing circuit 302, and can include computer code for executing (e.g., by processor 306) one or more processes described herein.

While shown as individual components, it will be appreciated that processor 306 and/or memory 310 can be implemented using a variety of different types and quantities of processors and memory. For example, processor 306 may represent a single processing device or multiple processing devices. Similarly, memory 310 may represent a single memory device or multiple memory devices. Additionally, in some embodiments, user device 302 may be implemented within a single computing device (e.g., one server, one housing, etc.). In other embodiments user device 302 may be distributed across multiple servers or computers (e.g., that can exist in distributed locations). For example, user device 302 may include multiple distributed computing devices (e.g., multiple processors and/or memory devices) in communication with each other that collaborate to perform operations.

Still referring to FIG. 3, memory 310 is shown to include a balance detection engine 312 configured to process sensor data and detect imbalances in a user’s movements based on the sensor data. In particular, balance detection engine 312 may receive data from left balance detector 324, right balance detector 326, and an inertial measurement unit (IMU) 318. As described above with respect to FIG. 1, the data received from left balance detector 324 and right balance detector 326 may include gyroscopic data and/or accelerometer data that indicates the orientation, angular velocity, acceleration, and/or movement of the user’s head due to the positioning of left balance detector 324 and right balance detector 326 in the user’s ears. In some embodiments, left balance detector 324 and right balance detector 326 may include individual IMUs which provide combined specific force, angular rate, and orientation data.

Likewise, IMU 318 may provide data that includes a specific force, angular rate, and/or orientation of user device 302. However, it should be appreciated that, in some embodiments, user device 302 may include individual gyrometers and/or accelerometers that provide specific force, angular rate, and/or orientation data (e.g., rather than a combined IMU 318). In any case, the data provided by IMU 318 can indicate a position and/or movement of the user’s body since, as mentioned above, user device 302 is generally carried by the user (e.g., in a pocket). Accordingly, the data received from IMU 318 may be used as reference or “baseline” values.

In some embodiments, the data received from any of left balance detector 324, right balance detector 326, and IMU 318 may be stored in a database 316 of memory 310. In general, database 316 may be any type of database (e.g., a table) or portion of memory 310 configured to maintain data for a period of time. In some embodiments, balance detection engine 312 can stored received data in database 316 for later retrieval. For example, data from left balance detector 324 and right balance detector 326 may be collected and stored over time to track the user’s movements.

In some embodiments, balance detection engine 312 may compare the data received from left balance detector 324 and right balance detector 326 (e.g., indicating a position and movement of the user’s head), also referred to herein as “head movement data,” with the data received from IMU 318 (e.g., indicating a position and movement of the user’s body), also referred to herein as “body movement data,” in order to detect imbalances in the user’s posture or movements. Specifically, balance detection engine 312 may use the baseline values collected by IMU 318 to calculate a center position of the user and, in some cases, may use time series data (e.g., data collected at discrete time steps over a time period) from IMU 318 to calculate a trajectory of the user. For example, balance detection engine 312 may use data from IMU 318 to determine the user’s body position (e.g., sitting, standing, etc.) and movement (e.g., the direction the user is walking, etc.).

In conjunction, balance detection engine 312 may compare the head movement data to the body movement data to identify imbalances. Additionally, balance detection engine 312 may detect when the user is performing an activity that may increase or aggravate BPPV or vertigo symptoms. For example, balance detection engine 312 may detect, based on one or both of the head and body movement data, that the user is performing high-intensity exercise or is traveling over a rough surface, which can cause an increase in BPPV symptoms. As another example, balance detection engine 312 may detect that the user has been laying down or, more generally, keeping their head in a constant position for an extended period of time, which can also trigger symptoms.

In some embodiments, balance detection engine 312 executes a machine learning model (e.g., a neural network) or another type of algorithm to calculate the user’s center position, which may change over time (e.g., as the user moves). For example, balance detection engine 312 may feed head and/or body movement data into the machine learning model, which can output an indication of whether an imbalance is detected. In some embodiments, the machine learning model is configured to “learn” the user’s movements over time. For example, balance detection engine 312 may track the user’s movements during periods where the user is known to not be experiencing an imbalance, and this known good data can be used to train the machine learning model. Subsequently, the model can be continuously executed using new head and body movement data to determine if the user’s movements are atypical, which can indicate an imbalance.

In some embodiments, balance detection engine 312 initiates one or more automated response actions if an imbalance is detected. In some such embodiments, balance detection engine 312 causes speaker 112 (e.g., included in each of left balance detector 324 and right balance detector 326) to emit a tone and/or a voice prompt alerting the user to the imbalance. For example, if the user is drifting to one direction when walking, speaker 112 may emit a voice prompt that alerts the user accordingly (e.g., “You are drifting to the left.”). In some embodiments, balance detection engine 312 causes speaker 112 to emit ultrasonic pulses (e.g., above 20 kHz) that can break apart calcium crystals in the user’s inner ear, which can help to relive some balance-related symptoms of BPPV.

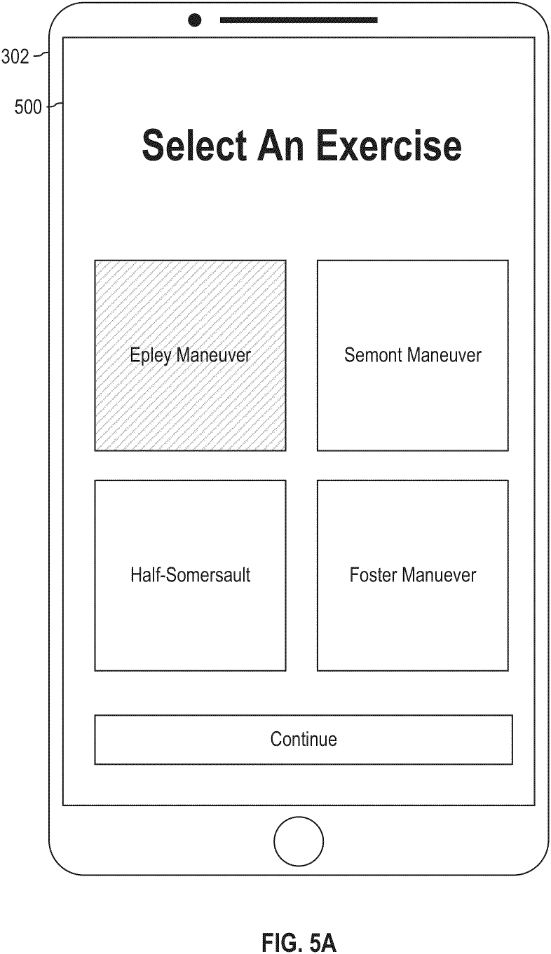

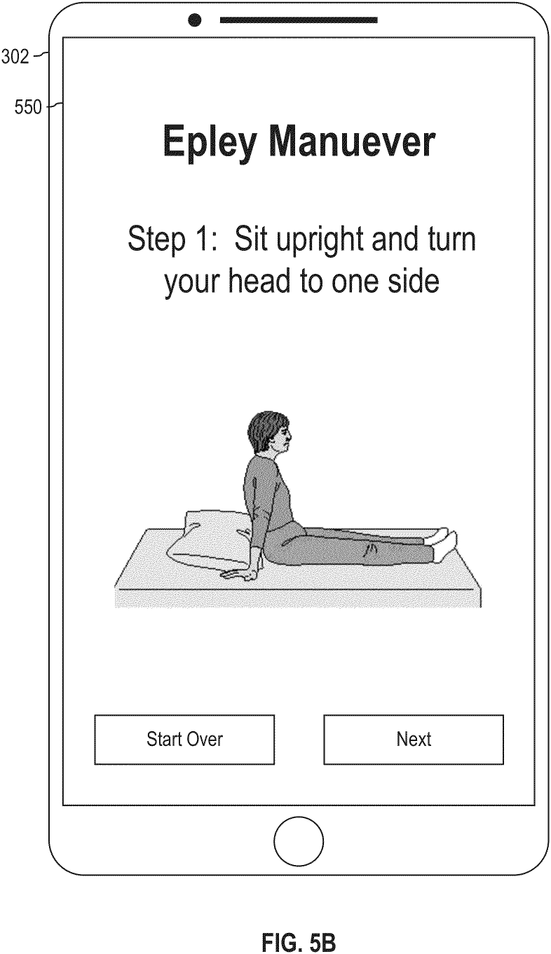

In some embodiments, balance detection engine 312 causes a user interface (UI) generator 314 to generate a graphical user interface (GUI) that alerts the user to the detected imbalance. In some such embodiments, the GUI may simply be or may include a notification to the user indicating the imbalance. In some embodiments, the GUI may include a number of recommended exercises or other corrective actions that the user can take to avoid or mitigate symptoms. For example, the GUI may prompt the user that they should stand up or change positions if they haven’t moved in a period of time. As another example, the GUI may provide a number of vertigo exercises, as shown in FIGS. 5A and 5B.

As a use-case scenario, a user of system 300 may have left and right balance detectors 324, 326 positioned in each of their left and right ears, respectively. As mentioned above, left and right balance detectors 324, 326 may be wirelessly connected to user device 302 via a short-range wireless connection such as Bluetooth^{®}. As the user walks (e.g., down a hallway), left and right balance detectors 324, 326 may continuously collect data indicating the positioning and movement of the user’s head. Simultaneously, user device 302 may continuously collect data indicate the positioning and movement of the user’s body and may continuously compare the collected body movement data to the collected head movement data. Based on this comparison, user device 302 can detect an imbalance, such as the user veering to the side, and may cause one or both of left and right balance detectors 324, 326 to a emit a sound or voice prompt alerting the user. Accordingly, the user may correct their trajectory or path to avoid running into objects, for example.

The GUIs generated by UI generator 314 may be displayed via a user interface 320. User interface 320 may include one or more components that allow a user to interact with user device 302. For example, user interface 320 may include at least one of a user input device and a display. Example user input devices can include a mouse, a keyboard, a keypad, a stylus, a touchscreen, or the like. Example displays can include LCD and/or LED displays, or even a simpler display comprising one or more lights. In some embodiments, user interface 320 includes a touch screen display that can both display GUIs and that can receive user inputs in the form of a touch.

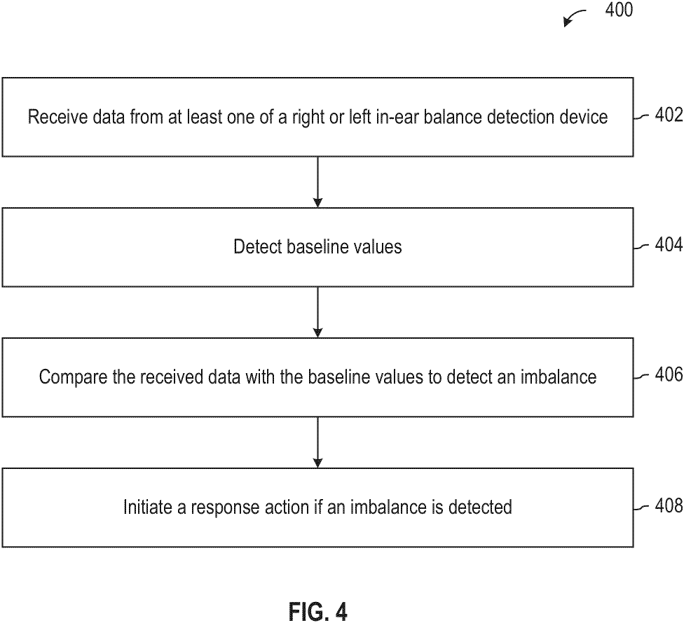

Referring now to FIG. 4, a flow diagram of a process 400 for detecting imbalances in a user’s movements is shown, according to some embodiments. As mentioned above, an imbalance may reference to any balance-related issue experienced by the user, such as drifting to one side when walking, imbalance when sitting or standing, dizziness, etc. Advantageously, process 400 may allow for the early detection of imbalances by tracking the user’s movements in real or near-real time. Accordingly, preventative or corrective actions can be implemented to mitigate BPPV and/or vertigo symptoms. In some embodiments, process 400 is implemented system 300, as described above, and more specifically may be implemented by user device 302. It will be appreciated that certain steps of process 400 may be optional and, in some embodiments, process 400 may be implemented using less than all of the steps.

As step 402, data is received from at least one of a right or left in-ear balance detection device. As described herein, the right and left in-ear balance detection devices are each the same as device 100 and/or left and right balance detectors 324, 326 described above. In general, the right in-ear balance detection device is worn or implanted in a right ear of a user and the left in-ear balance detection device is worn or implanted in a left ear of the user. Accordingly, for simplicity, the data received at step 402 may be generally referred to as “head movement data.” As also described above, the right and left in-ear balance detection devices may each be in wireless communication with a user device, such as the user’s smartphone, which receives said data for processing; however, it should also be understood that, in some embodiments, the right and left in-ear balance detection devices may include processors and memory and thus may be capable of collecting and processing data without transmitting the data to a remote device.

In some embodiments, the head movement data includes gyroscopic data and/or accelerometer data that indicates the orientation, angular velocity, acceleration, and/or movement of the user’s head. In some embodiments, the head movement data includes combined specific force, angular rate, and orientation data collected by an IMU in each of the right or left in-ear balance detection devices. In either case, because the right and left in-ear balance detection devices are worn or implanted in the user’s ears, the head movement data can be used to infer a position and/or movement of the user’s head.

At step 404, baseline values are detected. In particular, baseline values may be data collected by a second sensor unit or device carried on the user’s body, such as user device 302 of system 300 (e.g., carried in the user’s pocket, worn on the user’s wrist, etc.). As described above, user device 302 includes an IMU that can collect specific force, angular rate, and orientation data indicating a position and/or movement of the user’s body. In some other embodiments, baseline values are recorded by any other device that includes at least one of a gyroscope, an accelerometer, or an IMU, and that can be carried by the user. In some embodiments, the baseline values and/or the head movement data received at step 402 are stored in a database. For example, baseline values may be stored in the database for later analysis (e.g., as at step 406).

At step 406, the received head movement data is compared to the baseline values to detect an imbalance. In some embodiments, comparing the head movement data to the baseline (i.e., body movement) values includes calculating a center position of the user based on the baseline values. The center position may indicate, for example, the user’s stance, position, and/or posture, and/or may indicate the user’s center of gravity. In some cases, one or both of the head movement data and the baseline values are time series data, or data that is collected at discrete time steps over a time period. Time series data may be used at step 406 to further calculate a trajectory of the user. For example, the head movement data and/or baseline values may indicate the user’s body position (e.g., sitting, standing, etc.) and movement (e.g., the direction the user is walking, etc.) over time.

To this point, it should be appreciated that process 400 and/or the steps of process 400 (e.g., steps 402-406) may be performed continuously. For example, data may be continuously collected from right or left in-ear balance detection devices and/or the user device carried by the user, which can provide a robust data set indicating the user’s movements over time. Further, the head movement data and baseline values may be continuously compared to identify imbalances in real or near-real time.

In some embodiments, the baseline values are provided as inputs to a machine learning model (e.g., a neural network) or another type of algorithm to calculate the user’s center position, which may change over time (e.g., as the user moves). Similarly, in some embodiments, a machine learning model may be executed to compare the head movement data with the baseline values to identify imbalances. In some such embodiments, the machine learning model can be trained to recognize the user’s typical movements based on user movement data collected (i.e., tracked) over time. Thus, the trained machine learning model may identify atypical movements, which can indicate an imbalance, by analyzing the head movement data and/or baseline values.

In some embodiments, comparing the baseline values and head movement data can include determining when the user is performing an activity that may increase or aggravate BPPV or vertigo symptoms. For example, the data may indicate that the user is performing high-intensity exercise or is traveling over a rough surface, which can cause an increase in BPPV symptoms. As another example, it may be determined that the user has been laying down or, more generally, keeping their head in a constant position for an extended period of time, which can also trigger symptoms.

At step 408, if an imbalance is detected, a response action is automatically initiated. In some embodiments, the response action includes causing a speaker (e.g., speaker 112) included in each of the right and left in-ear balance detection devices to emit a tone and/or a voice prompt alerting the user to the imbalance. For example, if the user is drifting to one direction when walking, the speaker(s) may emit a voice prompt that alerts the user accordingly (e.g., “You are drifting to the left.”). In some embodiments, the response action includes causing the speaker(s) to emit ultrasonic pulses (e.g., above 20 kHz) that can break apart calcium crystals in the user’s inner ear, which can help to relive some balance-related symptoms of BPPV. In some embodiments, the response action includes generating and/or displaying a GUI that alerts the user to the detected imbalance. In some such embodiments, the GUI may simply be or may include a notification to the user indicating the imbalance. In some embodiments, the GUI may include a number of recommended exercises or other corrective actions that the user can take to avoid or mitigate symptoms. For example, the GUI may prompt the user that they should stand up or change positions if they haven’t moved in a period of time. As another example, the GUI may provide a number of vertigo exercises, as shown in FIGS. 5A and 5B.

Referring now to FIGS. 5A and 5B, example user interfaces that allow a user to select and perform various exercise for correcting vertigo are shown, according to some embodiments. Turning first to FIG. 5A, a first user interface 500 for selecting an exercise is shown. In some embodiments, interface 500 may be presented to a user responsive to the detection of an imbalance. In other embodiments, the user may access interface 500 manually to prevent the onset of symptoms. In any case, interface 500 is shown to include a plurality of graphical elements (e.g., buttons or icons) that can be selected by the user to start an associated exercise set. In this example, the user has selected the “Epley Maneuver” exercise set. Once an exercise set is selected the user may select a “Continue” icon to navigate to a second user interface 550, shown in FIG. 5B.

Interface 550 is shown to indicate a first step of the “Epley Maneuver” exercise set to the user. In other words, interface 550 may be a first slide, interface, or tile in a series of slides, interfaces, or tiles, each describing one step of the selected exercise set. In this regard, the user may “walk” through the exercise set in a step-by-step manner. In interface 550, for example, the user is instructed to “sit upright and turn [their] head to one side.” Interface 550 is also shown to include an illustration of the corresponding step/activity, which may be a picture, a video, etc. Once the user has completed the displayed step, one of a “Start Over” or “Next” button may be selected. The “Start Over” button may return the user to interface 500 if, for example, they cannot complete the exercise set. The “Next” button may navigate the user to a subsequent interface showing step two of the Epley Maneuver.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

What is claimed is:

1. A balance detection and correction system comprising:
at least one sensor positioned in an ear of a user;
a processor; and
a memory having instructions stored thereon that, when executed by the processor, causes the processor to perform operations comprising:
continuously tracking a position of the user’s head based on first data obtained from the first sensor;
comparing the position of the user’s head to second data obtained from a second sensor positioned on the user’s body to detect an imbalance in the user’s posture or movements; and
initiating a response action responsive to the detection of an imbalance.

2. The system of claim 1, wherein the at least one sensor is worn in the ear of the user or implanted in the ear of the user.

3. The system of claim 1, wherein the at least one sensor comprises at least one of a gyroscope, an accelerometer, or an inertial measurement unit (IMU).

4. The system of claim 1, wherein comparing the position of the user’s head to the second data comprises:
calculating a center position and a trajectory of the user based on the second data; and
comparing the first data to the center position and the trajectory to detect the imbalance.

5. The system of claim 1, further comprising a speaker positioned in the ear of the user, wherein the speaker and the at least one sensor are contained within a single housing.

6. The system of claim 5, wherein the response action comprises emitting, by the speaker, at least one of a tone or a voice prompt that indicates to the user that the imbalance is detected.

7. The system of claim 5, wherein the response action comprises emitting, by the speaker, ultrasonic sounds waves configured to break apart calcium crystals in the user’s ear.

8. The system of claim 1, wherein the response action comprises causing a user device associated with the user to display a notification.

9. The system of claim 1, wherein the response action comprises causing a user device associated with the user to display a prompt to perform one or more exercises for correcting the imbalance.

10. A method of detecting imbalances in human motion, the method comprising continuously:
receiving first data from a first sensor worn or implanted in an ear of a user, the first data indicating a position and motion of the user’s head;
receiving second data from a second sensor positioned on the body of the user, the second data indicating a position and motion of the user’s body;
comparing the first data and the second data to detect an imbalance in the user’s movements; and
initiating a response action responsive to the detection of an imbalance.

11. The method of claim 10, wherein comparing the first and the second data further comprises:
calculating a center position and a trajectory of the user based on the second data; and
comparing the first data to the center position and the trajectory to detect an imbalance.

12. The method of claim 10, wherein each of the first sensor and the second sensor comprise at least one of a gyroscope, an accelerometer, or an inertial measurement unit (IMU).

13. The method of claim 10, wherein the response action comprises emitting, by a speaker positioned in the user’s ear, at least one of a tone or a voice prompt that indicates to the user that the imbalance is detected.

14. The method of claim 10, wherein the response action comprises emitting, by a speaker positioned in the user’s ear, ultrasonic sounds waves configured to break apart calcium crystals in the user’s ear.

15. The method of claim 10, wherein the response action comprises causing a user device associated with the user to display a notification.

16. The method of claim 10, wherein the response action comprises causing a user device associated with the user to display a prompt to perform one or more exercises for correcting the imbalance.

17. An in-ear balance detection device comprising:
a gyroscope for detecting an orientation of a user’s head;
an accelerometer for detecting movement of the user’s head;
a wireless transceiver configured to wirelessly communicate data with a remote processing device, wherein the wireless transceiver transmits data from the gyroscope and the accelerometer, and wherein the wireless transceiver receives control signals from the remote processing device responsive to detection of an imbalance in the user’s movements based on the data from the gyroscope and the accelerometer; and
a speaker configured to emit auditory alerts responsive to a received control signal, wherein the auditory alerts indicate to the user that an imbalance is detected.

18. The device of claim 17, wherein the device is worn in an ear of the user.

19. The device of claim 17, wherein the device implanted in the ear of the user.

20. The device of claim 17, further comprising an inertial measurement unit (IMU), wherein the gyroscope and the accelerometer are part of the IMU.
